(19) United States
(12) Patent Application Publication
(10) Pub. No.: US 2012/0253160 A1
(43) Pub. Date: Oct. 4, 2012

(54) Methods and Reagents for in Vivo Imaging of Cancel Cell Lines

(75) Inventors: John Mauro, Eugene, OR (US); Thomas Steinberg, Eugene, OR (US); Julie Nyhus, Eugene, OR (US)

(73) Assignee: LIFE TECHNOLOGIES CORPORATION, Carlsbad, CA (US)

(21) Appl. No.: 13/439,279

(22) Filed: Apr. 4, 2012

Related U.S. Application Data
(63) Continuation of application No. 12/293,982, filed on Feb. 27, 2009, now abandoned.
(60) Provisional application No. 60/743,718, filed on Mar. 23, 2006.

(51) Int. Cl. A61B 5/1455

(52) U.S. Cl. 600/341

(57) ABSTRACT

Provided is reagents and methods for non-invasive in vivo imaging wherein the reagents comprise targeted carrier molecules conjugated to a NIB reporter molecule. In one aspect the targeted carrier molecule is an antibody, or fragment thereof that has specificity for an antigen in a living body, animal or human. In one embodiment the antibodes are anti-cancer/tumor marker antibodies, organ specific antibodies, tissue specific antibodies, cell type specific antibodies, cell surface specific antibodies, anti-viral antibodies, anti-bacterial antibodies and anti-pathogenic antibodies. The NIP reporter molecules are any fluorescent reporter molecule compatible with in vivo imaging and generally having an excitation wavelength of at least 580 nm.

### CROSS REFERENCE TO RELATED APPLICATIONS

This application claims priority to U.S. Provisional Application No. 60/743,718, filed Mar. 23, 2006, the disclosure of which is incorporated herein by reference in its entirety.

### FIELD OF THE INVENTION

The present invention relates to targeted carrier molecule near infrared (NIR) conjugates for in vivo imaging. The invention has applications in the fields of cell biology, in vivo imaging, pathology, neurology, immunology, proteomics and biosensing.

### BACKGROUND OF THE INVENTION

Carcinoembryonic antigen (CEA) is a glycoprotein that has been identified as a tumor marker in a number of human cancers, including colon, breast, pancreas, and lung tumors. This protein, first identified as an oncofetal antigen on the basis of its abundance in fetal gut and in adult colonic tumors, is a member of a multigene family involved in intercellular adhesion and migration. CEA is present in trace amounts in normal adult tissue, but malignancy is characterized by gross over-expression of this protein. CEA serum levels have had utility in diagnosis and surveillance of clinical progression of human colon cancer. (Pignatelli M, Durbin H, & Bodmer W F. Carcinoembryonic antigen functions as an accessory adhesion molecule mediating colon epithelial cell-collagen interactions. Proc. Natl. Acad. Sci. USA 1990; 87:1541-1545; Sanders D S A, Kerr, M A. Lewis blood group and CEA related antigens; coexpressed cell-cell adhesion molecules with roles in the biological progression and dissemination of tumors. J. Clin. Pathol.: Mol. Pathol. 1999; 52:174-178; Gold P and Goldenberg N A. The Carcinoembryonic Antigen (CEA): Past, Present, and Future. Perspectives in Colon and Rectal Surgery 1996; 9(2))

CEA has traditionally been used as a blood marker for cancer. In fact the first success in developing a blood test for a common cancer was in 1965, when carcinoembryonic antigen (CEA) was found in the blood of patients with colon cancer. The marker is too non-specific to be used as a screening for cancer, but has value as a predictor of tumor recurrence and in direct tumor imaging. CEA is expressed by about 80% of various cancer types, including colorectal, breast, lung and ovarian cancers.

CEA has been the focus of intensive pre-clinical investigations concerning detection and treatment of primary and metastatic colon cancer. In particular, animal models, especially mouse tumor models, have been the basis for development of therapies now in clinical trials. Thus CEA has utility both as a clinical disease marker and as a therapeutic target. (Paganelli G, Magnani, P, Zito, F, Villa, E, Sudati, F, Lopalco, L, Rosetti, C, Malcovati, M, Chiolerio, F, Seccamini, E. Three-step monoclonal antibody tumor targeting in carcinoembryonic antigen-positive patients. Cancer Res. 1991; 51: 5960-5966; Vogel C A, Galmiche M C, Westermann P, Sun L Q, Pelegrin A, Folli S, Bischof Delaloye A, Slosman D O, Mach, J P, Buchegger F. Carcinoembryonic antigen expression, antibody localization and immunodetection of human colon cancer liver metastases in nude mice: a model for radioimmunotherapy. Int. J. Cancer 1996; 67: 294-302; Nolan K F, Yun C-O, Akamatsu Y, Murphy J C, Leung S-O, Beecham, E J, Junghans, RP Bypassing immunization: optimized design against carcinoembryonic antigen (CEA)-expressing tumors, and lack of suppression by soluble CEA. Clin. Cancer Res. 1999; 5: 3921-3941; Armstrong AC and Hawkins RE Vaccines in oncology: background and clinical potential. Brit. J. Radiol. 2001 74: 991-1002; Ko B K, Kawano K, Murray J L, Disis M L, Efferson, CL Clinical studies of vaccines targeting breast cancer Clin. Cancer Res. 2003; 9: 3222-3234; Saha A, Chatterjee S K, Foon, K A, Primus F J, Sreedharan S, Mohanty K, Bhattacharya-Chatterjee M. Dendritic cells pulsed with an anti-idiotype antibody mimicking carcinoembryonic antigen (CEA) can reverse immunological tolerance to CEA and induce antitumor immunity in CEA transgenic mice Cancer Res. 2004; 64: 4995-5003).

The LS174T human colon cancer xenografts in nude mice model has been used extensively in tumor imaging and CEA biodistribution studies. The LS174T tumor cells hyperexpress the antigen on their surface and release the antigen into the media (1944 ng per 10(6) cells in 10 days). For imaging studies, the CEA monoclonal antibody is most often conjugated to a radionuclide (e.g. ¹¹¹In, Tc-99m,¹²³I) and detected with a gamma-scintillation camera. ImmunoPET, antibody-based targeting of a positron-emission tomography (PET) isotope selectively to cancer cells and single-photon emission computed tomography (SPECT) have been employed to increase signal/background image contrast, improving the sensitivity of detection.

CEA-based radioimaging is currently used on humans in the clinic. The Tc-99m radiolabeled Anti-CEA (CEA Scan) is approved for the detection of primary and recurrent colorectal cancer. CEA-Scan is not specific for colorectal carcinomas, since CEA is expressed by other carcinomas including cancers of the digestive system (oesophageal, gastric, pancreatic, and bile duct tumors), medullary thyroid cancer, and carcinomas of the lung, breast, ovary, endometrium and cervix.

Fluorescence based imaging has only been used in biopsied tissues or with endoscopy due to the lack of tissue penetration for fluorescent molecules that emit below the NIR range. Gastric adenocarcinomas in resected stomachs have been examined using endoscopic immunofluorescent techniques using fluorescein isothiocyanate (FITC)-labeled antibodies to carcinoembryonic antigen (CEA).

Thus, there is a need in the art for an antibody that recognizes CEA and can be visualized in a whole animal or human. There is also a need for other targeted antibodies conjugated to NIR for in vivo imaging. Detection of probe fluorescence in the near infrared (NIR) range of the electromagnetic spectrum represents an especially promising in vivo imaging modality as body tissues tend to be relatively transparent in this range (B. C. Wilson, Optical properties of tissues. Encyclopedia of Human Biology, 1991, 5, 587-597). Herein we provide anti-cancer/tumor marker antibodies, and other targeted carrier molecules, conjugated to a NIR fluorescent dye useful for in vivo imaging.

### SUMMARY OF THE INVENTION

In one embodiment is provided targeted carrier molecules conjugated to a NIR reporter molecule and methods for using. In one aspect the targeted carrier molecule is an antibody, or fragment thereof that has specificity for an antigen in a living body, animal or human. In one embodiment the antibodies are anti-cancer/tumor marker antibodies, organ specific antibodies, tissue specific antibodies, cell type specific antibodies, cell surface specific antibodies, anti-viral antibodies, anti-bacterial antibodies and anti-pathogenic antibodies.

The NIR reporter molecules are any fluorescent reporter molecule compatible with in vivo imaging and generally having an excitation wavelength of at least 580 nm. Reporter molecules include fluorescent organic dyes and particles. The reporter molecules comprise a reactive group and are conjugated to the present targeted carrier molecules using methods well known in the art.

The targeted carrier molecules reporter molecule conjugates are used for non-invasive in vivo imaging by inducing the conjugates into a living body where they are carried to the target site by circulating blood and lymph fluids. At any time after being introduced into the body, typically by injection into a vein, the living body is illuminated and imaged using instruments for in vivo imaging.

In another embodiment is provided kits for in vivo imaging comprising any combination of targeted carrier molecules reporter molecule conjugates, targeted carrier molecules, NIR reporter molecule, instructions for in vivo imaging and instructions for conjugating the reporter molecule to the targeted carrier molecule.

Methods of manufacturing compositions and kits described herein are provided and contemplated to fall within the scope of the invention as is the use of the compositions in methods for manufacturing imaging agents for use in the methods of the invention.

Further embodiments of the invention include those described in the detailed description.

### BRIEF DESCRIPTION OF THE FIGURES

FIG. 1: Shows a tumor labeled with CEA Ab-Alexa Fluor 750 conjugate imaged with a CRi Maestro Imaging System (Ex: 740 nm; Em: 790-950 nm).

FIG. 2: Shows a time course of tumor labeling by CEA Ab-Alexa Fluor 750 conjugate, wherein the signal refers to average intensity of the labeling in the tumor; background refers to signal from the mouse body next to the tumor.

FIG. 3: Shows Specificity of tumor labeling with CEA Ab-Alexa Fluor 750 conjugate imaged with CRi Maestro Imaging System (Ex: 740 nm; Em: 790-950 nm). Left: CEA+ LS174T tumor bearing nu/nu mouse. Right: CEA-SW620 tumor bearing nu/nu mouse.

FIG. 4: Shows three distinct sources of fluorescence following spectral unmixing of the image cube using the Nuance™ Software. The transferrin, tumor antigen, and gut content signals can be clearly separated (FIG. 4A). In a color diagram the transferrin showed as green, tumor antigen as red, and gut contents as blue signals. FIG. 4B shows the signal from only the anti-CEA dye conjugate.

### DETAILED DESCRIPTION OF THE INVENTION

Introduction

The present invention provides novel target specific carrier molecule-NIR reporter molecule conjugates for in vivo imaging. These dye conjugates are antibodies or other targeted proteins or peptides specific for a target or antigen in a living body that has been conjugated with a fluorescent dye(s) having an excitation wavelength compatible with in vivo imaging, typically about 580 nm to about 800 nm. The target specific dye conjugates travel relatively freely within the circulating blood until their preferential sequestration occurs at a target pathological or non-pathological tissue sites such as a diseased or injury tissue sites.

Definitions

Before describing the present invention in detail, it is to be understood that this invention is not limited to specific compositions or process steps, as such may vary. It must be noted that, as used in this specification and the appended claims, the singular form “a”, “an” and “the” include plural referents unless the context clearly dictates otherwise.

Unless defined otherwise, all technical and scientific terms used herein have the same meaning as commonly understood by one of ordinary skill in the art to which this invention is related. The following terms are defined for purposes of the invention as described herein.

Certain compounds of the present invention can exist in unsolvated forms as well as solvated forms, including hydrated forms. In general, the solvated forms are equivalent to unsolvated forms and are encompassed within the scope of the present invention. Certain compounds of the present invention may exist in multiple crystalline or amorphous forms. In general, all physical forms are equivalent for the uses contemplated by the present invention and are intended to be within the scope of the present invention.

The term “antibody” as used herein refers to a protein of the immunoglobulin (Ig) superfamily that binds noncovalently to certain substances (e.g. antigens and immunogens) to form an antibody-antigen complex, including but not limited to antibodies produced by hybridoma cell lines, by immunization to elicit a polyclonal antibody response, by chemical synthesis, and by recombinant host cells that have been transformed with an expression vector that encodes the antibody. In humans, the immunoglobulin antibodies are classified as IgA, IgD, IgE, IgG, and IgM and members of each class are said to have the same isotype. Human IgA and IgG isotypes are further subdivided into subtypes IgA₁, and IgA₂, and IgG₁, IgG₂, IgG₃, and IgG₄. Mice have generally the same isotypes as humans, but the IgG isotype is subdivided into IgG₁, IgG₂ₐ, IgG_{2b}, and IgG₃ subtypes. Thus, it will be understood that the term “antibody” as used herein includes within its scope (a) any of the various classes or sub-classes of immunoglobulin, e.g., IgG, IgM, IgE derived from any of the animals conventionally used and (b) polyclonal and monoclonal antibodies, such as murine, chimeric, or humanized antibodies. Antibody molecules have regions of amino acid sequences that can act as an antigenic determinant, e.g. the Fc region, the kappa light chain, the lambda light chain, the hinge region, etc. An antibody that is generated against a selected region is designated anti-[region], e.g. anti-Fc, anti-kappa light chain, anti-lambda light chain, etc. An antibody is typically generated against an antigen by immunizing an organism with a macromolecule to initiate lymphocyte activation to express the immunoglobulin protein. The term antibody, as used herein, also covers any polypeptide or protein having a binding domain that is, or is homologous to, an antibody binding domain, including, without limitation, single-chain Fv molecules (scFv), wherein a VH domain and a VL domain are linked by a peptide linker that allows the two domains to associate to form an antigen binding site (Bird et al., Science 242, 423 (1988) and Huston et al., Proc. Natl. Acad. Sci. USA 85, 5879 (1988)). These can be derived from natural sources, or they may be partly or wholly synthetically produced.

The term “antibody fragments” as used herein refers to fragments of antibodies that retain the principal selective binding characteristics of the whole antibody. Particular fragments are well-known in the art, for example, Fab, Fab′, and F(ab′)₂, which are obtained by digestion with various proteases and which lack the Fc fragment of an intact antibody or the so-called “half-molecule” fragments obtained by reductive cleavage of the disulfide bonds connecting the heavy chain components in the intact antibody. Such fragments also include isolated fragments consisting of the light-chain-variable region, “Fv” fragments consisting of the variable regions of the heavy and light chains, and recombinant single chain polypeptide molecules in which light and heavy variable regions are connected by a peptide linker. Other examples of binding fragments include (i) the Fd fragment, consisting of the VH and CH1 domains; (ii) the dAb fragment (Ward, et al., Nature 341, 544 (1989)), which consists of a VH domain; (iii) isolated CDR regions; and (iv) single-chain Fv molecules (scFv) described above. In addition, arbitrary fragments can be made using recombinant technology that retains antigen-recognition characteristics.

The term “aqueous solution” as used herein refers to a solution that is predominantly water and retains the solution characteristics of water. Where the aqueous solution contains solvents in addition to water, water is typically the predominant solvent.

An antigen is “associated with cancer” if it is abnormally expressed or present in detectable concentrations in cancer cell lines as compared with healthy, non-cancerous cells. Generally, antigens that are “associated with cancer” are over expressed in cancer cells and tumors.

The term “dye conjugate” refers to a dye molecule bound covalently or non-covalently to another carrier molecule, preferably an antibody and preferably the dyes are bound covalently. The dye conjugate can be directly bound through a single covalent bond, cross-linked or bound through a linker, such as a series of stable covalent bonds incorporating 1-20 nonhydrogen atoms selected from the group consisting of C, N, O, S and P that covalently attach the fluorescent dye to the antibody or another moiety such as a chemically reactive group or a biological and non-biological component. The conjugation or linker may involve a receptor binding motif, such as biotin/avidin.

The term “detectable response” as used herein refers to a change in or an occurrence of, a signal that is directly or indirectly detectable either by observation or by instrumentation and the presence or magnitude of which is a function of the presence of a target in the test sample. Typically, the detectable response is an optical response resulting in a change in the wavelength distribution patterns or intensity of absorbance or fluorescence or a change in light scatter, fluorescence quantum yield, fluorescence lifetime, fluorescence polarization, a shift in excitation or emission wavelength or a combination of the above parameters. The detectable change in a given spectral property is generally an increase or a decrease. However, spectral changes that result in an enhancement of fluorescence intensity and/or a shift in the wavelength of fluorescence emission or excitation are also useful.

The term “fluorophore” as used herein refers to a composition that is inherently fluorescent. Fluorophores may be substituted to alter the solubility, spectral properties or physical properties of the fluorophore. Numerous fluorophores are known to those skilled in the art and include, but are not limited to coumarin, acridine, furan, dansyl, cyanine, pyrene, naphthalene, benzofurans, quinolines, quinazolinones, indoles, benzazoles, borapolyazaindacenes, oxazine and xanthenes, with the latter including fluoresceins, rhodamines, rosamine and rhodols as well as other fluorophores described in RICHARD P. HAUGLAND, MOLECULAR PROBES HANDBOOK OF FLUORESCENT PROBES AND RESEARCH CHEMICALS (9^{th} edition, including the CD-ROM, September 2002). As used herein fluorophores of the present invention are compatible with in vivo imaging, optically excited in tissue, and generally have an excitation wavelength of about 580 nm to about 800 nm or longer.

The term “illuminating” as used herein refers to the application of any energy or light source, particularly near-infrared (NIR) and visible light, capable of exciting the dye conjugates of the invention.

The term “in vivo imaging” as used herein refers to methods or processes in which the structural, functional, or physiological state of a living being is examinable without the need for life ending sacrifice.

The term “kit” as used refers to a packaged set of related components, typically one or more compounds or compositions.

A “living body” includes any animal, such as a human, monkey, rat, mouse, dog, or cat that is alive.

The term “microsphere or microparticle” as used herein refers to particles of a size typically measured in the range from about 0.01 to about 10 microns and composed of any organic or inorganic material whose chemical and physical properties allow formation of functionally stable particles in this size range, which are preferably amenable to staining or association with a NIR dye. Preferred microspheres are polymeric organic particles, and can be comprised of a block copolymer. Some preferred microspheres for use in optical imaging of disease states are describe in PCT/US2006/061792, filed on Dec. 8, 2006, the contents of which are incorporated by reference as if set forth fully herein. In particular, compositions described in the contrast reagent section of PCT/US2006/061792 are contemplated for use as conjugates described herein.

The term “near IR dye” or “near IR reporter molecule” or “NIR dye” or “NIR reporter molecule” as used herein indicates a dye or reporter molecule with an excitation wavelength of about 580 nm to about 800 nm. Preferably, the NIR dyes emit in the range of about 590 nm to about 860 nm. Most preferred NIR dyes are excited from about 680 to about 790 nm. Preferred dyes include, Alexa Fluor 660 Dye, Alexa Fluor 680 dye, Alexa Fluor 700 dye, Alexa Fluor 750 dye, and Alexa Fluor 790 dye. The NIR dyes are particularly advantageous for in vivo imaging because they can be selectively visualized without exciting endogenous materials present in living body. Some of the NIR dyes have a large stokes shift, such that the excitation and emission wavelengths are separated by at least 20, 30, 40, 50, 60, 70 or 80 nm.

The term “non invasive in vivo imaging” as used herein refers to methods or processes in which the structural, functional, or physiological state of a being is examinable by remote physical probing without the need for breaching the physical integrity of the outer (skin) or inner (accessible orifices) surfaces of the body.

The terms “protein” and “polypeptide” are used herein in a generic sense to include polymers of amino acid residues of any length. The term “peptide” as used herein refers to a polymer in which the monomers are amino acids and are joined together through amide bonds, alternatively referred to as a polypeptide. When the amino acids are α-amino acids, either the L-optical isomer or the D-optical isomer can be used. Additionally, unnatural amino acids, for example, β-alanine, phenylglycine and homoarginine are also included. Commonly encountered amino acids that are not gene-encoded may also be used in the present invention. All of the amino acids used in the present invention may be either the D- or L-isomer. The L-isomers are generally preferred. In addition, other peptidomimetics are also useful in the present invention. For a general review, see, Spatola, A. F., in Chemistry and Biochemistry of Amino Acids, Peptides and Proteins, B. Weinstein, eds., Marcel Dekker, New York, p. 267 (1983).

The term “target antigen” as used herein indicates the particle of interest for the imaging methods described herein. Preferably, the antigen is implicated in a cancer pathway or over expressed in cancer cell lines. One particularly, preferred target antigen is CEA (Carcinoembryonic antigen).

In Vivo Imaging reagents

In general, for ease of understanding the present invention, the in vivo imaging reagents will first be described in detail, followed by the many and varied methods in which the in vivo imaging reagents find uses, which is followed by exemplified methods of use.

Provided are in vivo imaging reagents, comprising a targeting carrier molecule and a fluorescent reporter molecule suitable for in vivo imaging, and methods for using in methods of non-invasive in vivo imaging. The targeting carrier molecules are conjugated to the present fluorescent dyes and once introduced into the body, animal, insect or human, relatively freely within the circulating blood until encountering the target epitope wherein preferential sequestration of the conjugated targeted carrier molecule occurs.

The targeted carrier molecule is any biological or non-biological molecule that has a specific binding partner found in the body and can travel relatively freely in circulating blood and lymph. A specific binding partner is a molecule that selectively binds non-covalently with the targeted carrier molecule. Examples include, but are not limited to, antibodies and antigens. Examples of antibodies include, but are not limited to, anti-cancer/tumor marker antibodies, organ specific antibodies, tissue specific antibodies, cell type specific antibodies, cell surface specific antibodies, anti-viral antibodies, anti-bacterial antibodies and anti-pathogenic antibodies. Wherein these antibodies react with an antigen that occurs exclusively on a organ, tissue, or cell to distinguish it from other organ, tissue, or cell types. For example, two types of organ, or tissue, specificity have been proposed: (1) first-order or tissue specificity is attributed to the presence of an antigen characteristic of a particular organ in a single species; (2) second-order organ specificity is attributed to an antigen characteristic of the same organ in many, even unrelated species.

### Anti-Cancer/Tumor Marker Antibodies

α₂-Macroglobulin
α-Fetoprotein (AFP)
β₂-Microglobulin
β-Catenin
ACTH C terminal
ACTH N terminal
ACTR/AlB1
Alpha Fetoprotein
BCA-225
Bcl-2
BRCA2
Bromodeoxyuridine
CA 125
CA 15-3
CA 19-9
Calcitonin
Calretinin
Cathepsin D
CD15
CD63
CD74
CEA (Carcinoembryonic Antigen)
Chorionic gonadotropin (β-subunit) (βHCG)
Chromogranin A
c-Kit (CD117)
Cks1
Clathrin Antigen
Claudin-3
Claudin-4
Claudin-7
c-Met
c-Myc
Collagen Type IV
Collagen Type VII
COX-1
COX-2
Cyclin D1/D2 & D3 antibodies
Cyclin E
Cytokeratin (Acidic)
Cytokeratin (Basic)
Cytokeratin (HMW)
Cytokeratin 18
Cytokeratin 19
Cytokeratin 20
Cytokeratin ⅚
Cytokeratin 6
Cytokeratin 7
Cytokeratin 8
Cytokeratin 8/18
E2 F-1
E-Cadherin
EGFr
EGP2 (Epithelial Glycoprotein 2)
EMA (Epithelial Membrane Antigen)
EMMPRIN
Enolase
EphB4 Receptor
ER (Estrogen Receptor)
EZH2
Ezrin
FHIT
Galectin-1
Galectin-3
GCDFP-15
Glial Filament Acidic Protein
HER2 (c-erbB-2)
HER4
HPV Early Protein
HPV16 Late I Protein
Human Epithelial Proliferating Ag
Human Epithelium Specific Ag
Human Milk Fat Globule Membrane
Human Milk Fat Globulin (HMFG1)
Human Milk Fat Globulin (HMFG2)
Involucrin
JAB1
Ki-67
Lewis A Ag
LRP/MVP
Major Vault Protein
MAP Kinase (ERK1+ERK2)
MART-1 (Melan-A)
MDM2
Melanoma Associated Antigen
Melanosome
Metallothionein
MGMT
MLH1
MSH2
MSH6
MTA1
MUC1 (Mucin 1)
MUC2 (Mucin 2)
MUC5AC
N-Cadherin
Neu-Oncogene
Nitric Oxide Synthase
Nucleophosmin/B23
NY-ESO-1
Occludin
p16
p21 (WAF1/Cip1)
p27
p34
P53 Oncoprotein
Pancreatic Islet Cell Antibody
PARP
Paxillin
PD-ECGF
P-Glycoprotein (MDR)
phospho-MAP Kinase (ERK1+2)
Phosphotyrosine
Placental Alkaline Phosphatase
PR (Progesterone Receptor)
PRL-3
PRLr
Proliferating Cell Protein Ki-67
pS2
PSA (Prostate Specific Antigen)
PsAP (Prostatic Acid Phosphatase)
PTEN
PTTG-1 (Pituitary Tumor Transforming Gene-1)
Retinoblastoma Gene Product
SCLC (Small Cell Lung Cancer, CD56, N-CAM)
Sialyl Lewis A
SKP2
Smad3
STAT3
TAG-72 (CA 72.4)
TdT
Tenascin
Thyroglobulin
TIMP-2
Topo II
TS (Thymidylate Synthase)
TTF-1 (Thyroid Transcription Factor 1)
uPAR
Villine
Vmentin
Wt1 (Wilm's tumor)
ZO-1

One embodiment of the invention provides a composition comprising a dye conjugate comprising a NIR dye or reporter molecule and an antibody that binds to CEA (Carcinoembryonic Antigen).

Any fluorescent dye known to one of skill in the art having an excitation wavelength compatible with in vivo imaging can be used as a NIR reporter molecule for the above described target specific carrier molecules. Typically the fluorescent dyes will have an excitation wavelength of at least 580 nm. A wide variety of long wavelength fluorescent dyes that may be suitable for conjugation to proteins and peptides are already known in the art (RICHARD P. HAUGLAND, MOLECULAR PROBES HANDBOOK OF FLUORESCENT PROBES AND RESEARCH PRODUCTS (2002)) (Supra).

A fluorescent dye or fluorophore of the present invention is any chemical moiety that exhibits an absorption maximum beyond 580 nm and that is optically excited and observable in tissue. Dyes of the present invention include, without limitation; a pyrene, an anthracene, a naphthalene, an acridine, a stilbene, an indole or benzindole, an oxazole or benzoxazole, a thiazole or benzothiazole, a 4-amino-7-nitrobenz-2-oxa-1, 3-diazole (NBD), a carbocyanine (including any corresponding compounds in U.S. Ser. Nos. 09/968,401; 09/969,853 and 11/150,596 and U.S. Pat. Nos. 6,403,807; 6,348,599; 5,486,616; 5,268,486; 5,569,587; 5,569,766; 5,627,027; 6,664,047; 6,048,982 AND 6,641,798), a carbostyryl, a porphyrin, a salicylate, an anthranilate, an azulene, a perylene, a pyridine, a quinoline, a borapolyazaindacene (including any corresponding compounds disclosed in U.S. Pat. Nos. 4,774,339; 5,187,288; 5,248,782; 5,274,113; and 5,433,896), a xanthene (including any corresponding compounds disclosed in U.S. Pat. No. 6,162,931; 6,130,101; 6,229,055; 6,339,392; 5,451,343 and U.S. Ser. No. 09/922,333), an oxazine or a benzoxazine, a carbazine (including any corresponding compounds disclosed in U.S. Pat. No. 4,810,636), a phenalenone, a coumarin (including an corresponding compounds disclosed in U.S. Pat. Nos. 5,696,157; 5,459,276; 5,501,980 and 5,830,912), a benzofuran (including an corresponding compounds disclosed in U.S. Pat. Nos. 4,603,209 and 4,849,362) and benzphenalenone (including any corresponding compounds disclosed in U.S. Pat. No. 4,812,409) and derivatives thereof. As used herein, oxazines include resorufins (including any corresponding compounds disclosed in 5,242,805), aminooxazinones, diaminooxazines, and their benzo-substituted analogs.

Where the dye is a xanthene, the dye is optionally a fluorescein, a rhodol (including any corresponding compounds disclosed in U.S. Pat. Nos. 5,227,487 and 5,442,045), a rosamine or a rhodamine (including any corresponding compounds in U.S. Pat. Nos. 5,798,276; 5,846,737; 5,847,162; 6,017,712; 6,025,505; 6,080,852; 6,716,979; 6,562,632). As used herein, fluorescein includes benzo- or dibenzofluoresceins, seminaphthofluoresceins, or naphthofluoresceins. Similarly, as used herein rhodol includes seminaphthorhodafluors (including any corresponding compounds disclosed in U.S. Pat. No. 4,945,171). Fluorinated xanthene dyes have been described previously as possessing particularly useful fluorescence properties (Int. Publ. No. WO 97/39064 and U.S. Pat. No. 6,162,931).

In one embodiment the dye has an emission spectrum with its maximum greater than about 600 nm. In a further embodiment the dye or fluorophore has an emission spectrum with its maximum greater than about 620 nm, an emission maximum greater than about 650 nm, an emission maximum great than about 700 nm, an emission maximum greater than about 750 nm, or an emission maximum greater than about 800 nm. In one aspect the dye is a cyanine dye. Preferred are those dyes sold under the trade name Alexa Fluor® dye or spectrally similar dyes sold under the trade names Cy® dyes, Atto dyes or Dye dyes.

Preferred Alexa Fluor dyes include, Alexa Fluor 660 Dye, Alexa Fluor 680 dye, Alexa Fluor 700 dye, Alexa Fluor 750 dye, and Alexa Fluor 790 dye.

Typically the dye contains one or more aromatic or heteroaromatic rings, that are optionally substituted one or more times by a variety of substituents, including without limitation, halogen, nitro, sulfo, cyano, alkyl, perfluoroalkyl, alkoxy, alkenyl, alkynyl, cycloalkyl, arylalkyl, acyl, aryl or heteroaryl ring system, benzo, or other substituents typically present on chromophores or fluorophores known in the art.

In another embodiment, the present proteins or peptides can be conjugated with fluorescent or light scattering nanocrystals [Yguerabide, J. and Yguerabide, EE, 2001 J. Cell Biochem Suppl.37: 71-81; US Patent Nos. 6,214,560; 6,586,193 and 6,714,299]. These fluorescent nanocrystals can be semiconductor nanocrystals or doped metal oxide nanocrystals. Nanocrystals typically are comprised of a core comprised of at least one of a Group II-VI semiconductor material (of which ZnS, and CdSe are illustrative examples), or a Group III-V semiconductor material (of which GaAs is an illustrative example), a Group IV semiconductor material, or a combination thereof. The core can be passivated with a semiconductor overlayering (“shell”) uniformly deposited thereon. For example, a GroupII-VI semiconductor core may be passivated with a Group II-VI semiconductor shell (e.g., a ZnS or CdSe core may be passivated with a shell comprised of YZ wherein Y is Cd or Zn, and Z is S, or Se). Nanocrystals can be soluble in an aqueous-based environment. An attractive feature of semiconductor nanocrystals is that the spectral range of emission can be changed by varying the size of the semiconductor core.

After selection of an appropriate dye with the desired spectral characteristics, typically where the excitation wavelength is at least 580 nm, the dyes are conjugated to a targeted carrier moelcule, using methods well known in the art (Haugland, MOLECULAR PROBES HANDBOOK, supra, (2002)). Preferably, conjugation to form a covalent bond consists of simply mixing the reactive compounds of the present invention in a suitable solvent in which both the reactive compound and the substance to be conjugated are soluble. The reaction preferably proceeds spontaneously without added reagents at room temperature or below. For those reactive compounds that are photoactivated, conjugation is facilitated by illumination of the reaction mixture to activate the reactive compound. Chemical modification of water-insoluble substances, so that a desired compound-conjugate may be prepared, is preferably performed in an aprotic solvent such as dimethylformamide, dimethylsulfoxide, acetone, ethyl acetate, toluene, or chloroform. Similar modification of water-soluble materials is readily accomplished through the use of the instant reactive compounds to make them more readily soluble in organic solvents.

Preparation of peptide or protein conjugates typically comprises first dissolving the protein to be conjugated in aqueous buffer at about. 1-10 mg/mL at room temperature or below. Bicarbonate buffers (pH about 8.3) are especially suitable for reaction with succinimidyl esters, phosphate buffers (pH about 7.2-8) for reaction with thiol-reactive functional groups and carbonate or borate buffers (pH about 9) for reaction with isothiocyanates and dichlorotriazines. The appropriate reactive compound is then dissolved in a nonhydroxylic solvent (usually DMSO or DMF) in an amount sufficient to give a suitable degree of conjugation when added to a solution of the protein to be conjugated. The appropriate amount of compound for any protein or other component is conveniently predetermined by experimentation in which variable amounts of the compound are added to the protein, the conjugate is chromatographically purified to separate unconjugated compound and the compound-protein conjugate is tested in its desired application.

Following addition of the reactive compound to the component solution, the mixture is incubated for a suitable period (typically about 1 hour at room temperature to several hours on ice), the excess compound is removed by gel filtration, dialysis, HPLC, adsorption on an ion exchange or hydrophobic polymer or other suitable means. The compound-conjugate is used in solution or lyophilized. In this way, suitable conjugates can be prepared from antibodies, antibody fragments, and other targeting carrier molecules.

Conjugates of polymers, including biopolymers and other higher molecular weight polymers are typically prepared by means well recognized in the art (for example, Brinkley et al., Bioconjugate Chem., 3: 2 (1992)). In these embodiments, a single type of reactive site may be available, as is typical for polysaccharides) or multiple types of reactive sites (e.g. amines, thiols, alcohols, phenols) may be available, as is typical for proteins. Selectivity of labeling is best obtained by selection of an appropriate reactive dye. For example, modification of thiols with a thiol-selective reagent such as a haloacetamide or maleimide, or modification of amines with an amine-reactive reagent such as an activated ester, acyl azide, isothiocyanate or 3,5-dichloro-2,4,6-triazine. Partial selectivity can also be obtained by careful control of the reaction conditions.

When modifying polymers with the compounds, an excess of compound is typically used, relative to the expected degree of compound substitution. Any residual, unreacted compound or a compound hydrolysis product is typically removed by dialysis, chromatography or precipitation. Presence of residual, unconjugated dye can be detected by thin layer chromatography using a solvent that elutes the dye away from its conjugate. In all cases it is usually preferred that the reagents be kept as concentrated as practical so as to obtain adequate rates of conjugation.

Methods

The present invention provides methods for non-invasive in vivo imaging of a target antigen in a living animal or human body with the use of a targeted carrier molecule conjugated to a NIR dye or particle.

In one embodiment is provided a method for imaging a target antigen in a living body, wherein the method comprises;

a) providing a targeted carrier molecule dye conjugate wherein the conjugate comprises a NIR dye and a carrier molecule that is thought to bind to a target antigen in the body;

b) introducing the targeted carrier molecule dye conjugate into the body to form a contacted body;

c) non-invasively illuminating the contacted body with an appropriate wavelength to form an illuminated body, wherein the integrity of the body is not disrupted;

d) observing the illuminated body wherein the target antigen is imaged.

In a further embodiment, the target carrier molecule is an anti-cancer/tumor marker, such as any of the antibodies disclosed in table 1. In one aspect anti-CEA conjugated to a NIR dye is used to visualize tumor present in a live mouse.

Another embodiment of the invention provides a method for imaging a target antigen in a living body, wherein the method comprises;

a) providing a dye conjugate comprising a NIR dye and an antibody or antibody fragment that binds to the target antigen;

b) introducing the dye conjugate into the body to form a contacted body;

c) illuminating the contacted body with an appropriate wavelength to form an illuminated body; and

d) observing the illuminated body wherein the target antigen is imaged; wherein the target antigen is associated with cancer.

In a preferred embodiment, the target antigen is CEA (Carcinoembryonic Antigen). Alternatively, the target antigen is selected from the group consisting of α₂-Macroglobulin, α-Fetoprotein (AFP), β₂-Microglobulin, β-Catenin, ACTH C terminal, ACTH N terminal, ACTR/AIB1, Alpha Fetoprotein, BCA-225, Bcl-2, BRCA2, Bromodeoxyuridine, CA 125, CA 15-3, CA 19-9, Calcitonin, Calretinin, Cathepsin D, CD15, CD63, CD74, CEA (Carcinoembryonic Antigen), Chorionic gonadotropin (β-subunit) (βHCG), Chromogranin A, c-Kit (CD117), Cks1, Clathrin Antigen, Claudin-3, Claudin-4, Claudin-7, c-Met, c-Myc, Collagen Type IV, Collagen Type VII, COX-1, COX-2, Cyclin D1/D2 & D3, Cyclin E, Cytokeratin (Acidic or Basic), Cytokeratin (HMW), Cytokeratin 18, Cytokeratin 19, Cytokeratin 20, Cytokeratin ⅚, Cytokeratin 6, Cytokeratin 7, Cytokeratin 8, Cytokeratin 8/18, E2F-1, E-Cadherin, EGFr, EGP2 (Epithelial Glycoprotein 2), EMA (Epithelial Membrane Antigen), EMMPRIN, Enolase, EphB4 Receptor, ER (Estrogen Receptor), EZH2, Ezrin, FHIT, Galectin-1, Galectin-3, GCDFP-15, Glial Filament Acidic Protein, HER2 (c-erbB-2), HER4, HPV Early Protein, HPV16 Late I Protein, Human Epithelial Proliferating Ag, Human Epithelium Specific Ag, Human Milk Fat Globule Membrane, Human Milk Fat Globulin (HMFG1), Human Milk Fat Globulin (HMFG2), Involucrin, JAB1, Ki-67, Lewis A Ag, LRP/MVP, Major Vault Protein, MAP Kinase (ERK1+ERK2), MART-1 (Melan-A), MDM2, Melanoma Associated Antigen, Melanosome, Metallothionein, MGMT, MLH1, MSH2, MSH6, MTA1, MUC1 (Mucin 1), MUC2 (Mucin 2), MUC5AC, N-Cadherin, Neu-Oncogene, Nitric Oxide Synthase, Nucleophosmin/B23, NY-ESO-1, Occludin, p16, p21 (WAF1/Cip1), p27, p34, P53 Oncoprotein, Pancreatic Islet Cell Antibody, PARP, Paxillin, PD-ECGF, P-Glycoprotein (MDR), phospho-MAP Kinase (ERK1+2), Phosphotyrosine, Placental Alkaline Phosphatase, PR (Progesterone Receptor), PRL-3, PRLr, Proliferating Cell Protein Ki-67, pS2, PSA (Prostate Specific Antigen), PsAP (Prostatic Acid Phosphatase), PTEN, PTTG-1 (Pituitary Tumor Transforming Gene-1), Retinoblastoma Gene Product, SCLC (Small Cell Lung Cancer, CD56, N-CAM), Sialyl Lewis A, SKP2, Smad3, STAT3, TAG-72 (CA 72.4), TdT, Tenascin, Thyroglobulin, TIMP-2, Topo II, TS (Thymidylate Synthase), TTF-1 (Thyroid Transcription Factor 1), uPAR, Villine, Vmentin, Wt1 (Wilm's tumor), and ZO-1.

In a further embodiment, the introducing step is non-invasive such that the integrity of the body is not disrupted.

In another embodiment, the NIR dye has an excitation wavelength of about 580 nm to about 800 nm. More particularly, the NIR dye has an excitation wavelength of about 660 nm to about 790 nm. In another embodiment, the NIR dye has an emission wavelength of about 600 nm to about 850 nm.

In another embodiment, the NIR dye is selected from the group consisting of a pyrene, an anthracene, a naphthalene, an acridine, a stilbene, an indole or benzindole, an oxazole or benzoxazole, a thiazole or benzothiazole, a 4-amino-7-nitrobenz-2-oxa-1, 3-diazole (NBD), a carbocyanine, a carbostyryl, a porphyrin, a salicylate, an anthranilate, an azulene, a perylene, a pyridine, a quinoline, a borapolyazaindacene, a xanthene, an oxazine, a benzoxazine, a resorufin, a carbazine, a phenalenone, a coumarin, a benzofuran, a benzphenalenone and derivatives thereof. Alternatively, the NIR dye is a semiconductor nanocrystal.

In another embodiment, the antibody is a monoclonal antibody. More particularly, the antibody is specific for CEA (Carcinoembryonic Antigen).

In another embodiment, the living body is a non-human vertebrate. More particularly, the living body is a mouse or rat. Alternatively, the living body is a human.

In another embodiment, the cancer is cervical cancer, testicular tumor, lung carcinoma, small cell lung carcinoma, bladder carcinoma, epithelial carcinoma, glioma, colorectal carcinoma, pancreatic cancer, breast cancer, ovarian cancer, prostate cancer, squamous cell carcinoma, basal cell carcinoma, adenocarcinoma, renal cell carcinoma, hepatoma, bile duct carcinoma, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, rhabdosarcoma, sweat gland carcinoma, sebaceous gland carcinoma, papillary carcinoma, papillary adenocarcinomas, cystadenocarcinoma, medullary carcinoma, bronchogenic carcinoma, choriocarcinoma, seminoma, embryonal carcinoma, Wilms' tumor, astrocytoma, Kaposi's sarcoma, medulloblastoma, craniopharyngioma, ependymoma, pinealoma, hemangioblastoma, acoustic neuroma, oligodendroglioma, meningioma, neuroblastoma, retinoblastoma, myeloma, lymphoma, or leukemia.

In another embodiment, the introducing step comprises parenteral administration of the dye conjugate into the body. More particularly, the introducing step involves intravenous injection.

Another embodiment further comprises a step of:
incubating the contacted body for a period of time sufficient for the dye conjugate to contact the target antigen. More particularly, the period of time is at least 90 minutes.

Another embodiment further comprises a step of:
transmitting data onto a computer processor, wherein the data represents the illuminated body; and
performing an analysis of the data with the computer processor to determine a result indicating the presence, amount or location of the target antigen.

Another embodiment further comprises a display unit or printout which visually displays the result.

Another embodiment provides a method for identifying a tumor in a living body, the method comprising:
a) providing a dye conjugate comprising a NIR dye and an antibody that binds to a target antigen;
b) introducing the dye conjugate into the body to form a contacted body;
c) illuminating the contacted body with an appropriate wavelength to form an illuminated body; and
d) observing the illuminated body wherein the tumor is identified; wherein the target antigen is associated with cancer.

Another embodiment of the invention provides a method of manufacturing a dye conjugate for use in imaging a target antigen associated with cancer in a live body, wherein the dye conjugate comprises a NIR dye and an antibody that binds to the target antigen. In another embodiment, the target antigen is CEA (Carcinoembryonic Antigen).

The targeted dye conjugate can be introduced by any means known for uptake into the body, which includes, but is not limited to, orally or intravenously. In one aspect the targeted carrier molecule dye conjugate is introduced into the body intravenously by injection with a needle into a vein, such as the tail vein of a mouse or rat. Once in circulation, the targeted carrier molecule dye conjugate travels relatively freely until encountering the target antigen, wherein the conjugate associates non-covalently with the target and is sequestered in a specific part of the body until being cleared by normal bodily processes.

The living body may be illuminated at any time after the targeted carrier molecule dye conjugate has been introduced into the body. In one aspect the body is illuminated 15 min, 30 min, 90 min, 2 hr, 6 hr, 24 hr, 48 hr, 3 days, 4 days, 7 days or longer post-injection. The instrument used for illumination and visualization is any instrument known in the art for non-in vivo imaging.

Kits

Due to the advantageous properties and the simplicity of use of the instant targeted carrier molecule dye conjugate, they are particularly useful in the formulation of a kit for non-invasive in vivo imaging. In one embodiment the kits comprise instant targeted carrier molecule dye conjugates and instructions for in vivo imaging. In another embodiment the kits comprise a reactive NIR reporter molecule, dye or particle, a targeted carrier molecule, instructions for conjugating the reactive reporter molecule to the targeted carrier molecule and instructions for in vivo imaging. In yet another embodiment, the kits comprise a reactive reporter molecule, instructions for conjugating the reactive reporter molecule to a targeted carrier molecule and instructions for in vivo imaging.

One particular embodiment provides a kit for imaging a target antigen in a living body comprising:

a) a dye conjugate comprising a NIR dye and an antibody that binds to a target antigen associated with cancer; and

b) instructions for imaging the target antigen.

More particularly, the kit further comprises at least one of: a needle, imaging software, reagents, buffers, diluents, excipients, additional dyes or antibodies.

In a preferred embodiment of the kit, the target antigen is CEA (Carcinoembryonic Antigen).

### EXAMPLES

The examples below are given so as to illustrate the practice of this invention. They are not intended to limit or define the entire scope of this invention.

Example 1:Preparation of the monoclonal antibody: anti-CEA Alexa Fluor® 680

The mouse monoclonal antibody, anti-Carcinoembryonic Antigen (CEA), clone Col-1, (MPX18-0057, Invitrogen Corp, Carlsbad, Calif.) was conjugated with Alexa Fluor® 680 dye according to the procedure in the SAIVI™ Alexa Fluor® 680 Antibody/Protein 1 mg labeling kit (S30039, Invitrogen Corp, Eugene, Oreg.). Briefly, 500 μl of 2 mg/ml of antibody protein in PBS was combined with 50 μl 1 M sodium bicarbonate, pH 8.3, and 45 μl 15 mM lysine. This mixture was added to a reaction tube containing 96 μg lyophilized Alexa Fluor 680 carboxylic acid, succinimidyl ester. The reactive dye was dissolved and fully mixed, and the reaction was incubated for 60 minutes at ambient temperature (18-20° C.), protected from light. The dye-conjugated antibody was purified by size exclusion chromatography. The degree of labeling (mole fluorophore/mole antibody), determined spectrally, was 2.1

The dye conjugated antibody was adjusted to 1 mg/ml and filtered through an ELF® spin filter, 0.2 um pore size, (E6606, Invitrogen Corp., Eugene, Oreg.) that had been sanitized by prior filtration of 70% ethyl alcohol.

### Example 2

Preparation of the monoclonal antibody: anti-CEA Alexa Fluor® 750

The mouse monoclonal antibody, anti-Carcinoembryonic Antigen (CEA), clone Col-1, (MPX18-0057, Invitrogen Corp, Carlsbad, Calif.) was conjugated with Alexa Fluor® 750 dye according to the procedure in the SAIVI™ Alexa Fluor® 750 Antibody/Protein 1 mg labeling kit (S30040, Invitrogen Corp, Eugene, Oreg.). Briefly, 500 μl of 2 mg/ml of antibody protein in PBS was combined with 50 μl 1 M sodium bicarbonate, pH 8.3, and 40 μl 15 mM lysine. This mixture was added to a reaction tube containing 90 μg lyophilized Alexa Fluor 750 carboxylic acid, succinimidyl ester. The reactive dye was dissolved and fully mixed, and the reaction was incubated for 60 minutes at ambient temperature (18-20° C.), protected from light. The dye-conjugated antibody was purified by size exclusion chromatography. The degree of labeling (mole fluorophore/mole antibody), determined spectrally, was 1.6.

The dye conjugated antibody was adjusted to 1 mg/ml and filtered through an ELF® spin filter, 0.2 um pore size, (E6606, Invitrogen Corp., Eugene, Oreg.) that had been sanitized by prior filtration of 70% ethyl alcohol.

The monoclonal antibody, COL-1 described herein recognizes a restricted epitope on CEA expressed in neoplasms of epithelial origin, particularly those from the gastrointestinal tract, breast, lung, and bladder, but not in normal tissue. (Murarao R, Wunderlich D, Thor A, Lundy P, Noguchi P, Cunningham R, Schlom J. Definition by monoclonal antibodies of a repertoire of epitopes on carcinoembryonic antigen differentially expressed in human colon carcinomas versus normal adult tissues. Cancer Res. 1985; 45: 5769-5780; Zimmermann W, Weber B, Ortlieb B, Rudert F, Schempp W, Fiebig H H, Shively J E, von Kleist S, Thompson J A Chromosomal localization of the carcinoembryonic antigen gene family and differential expression in various tumors. Cancer Res. 1988; 48: 2550-2554; Shi Z R, Tacha D, Itzkowitz SH Monoclonal antibody COL-1 reacts with restricted epitopes on carcinoembryonic antigen: an immunohistochemical study. J. Histochem. Cytochem. 1994; 42: 1212-1219)

### Example 3

Preparation of the monoclonal antibody: anti-CEA Alexa Fluor® 790

The mouse monoclonal antibody, anti-Carcinoembryonic Antigen (CEA), clone Col-1, (MPX18-0057, Invitrogen Corp, Carlsbad, Calif.) was conjugated with Alexa Fluor® 790 dye in a procedure very similar to that of the SAIVI™ Alexa Fluor® dye Antibody/Protein labeling kits in the previous 2 examples. Briefly, 500 μl of 2 mg/ml of antibody protein in PBS was combined with 50 μl 1 M sodium bicarbonate, pH 8.3, and 40 μl 15 mM lysine. This mixture was added to a reaction tube containing 125 μg lyophilized Alexa Fluor 790 carboxylic acid, succinimidyl ester. The reactive dye was dissolved and fully mixed, and the reaction was incubated for 60 minutes at ambient temperature (18-20° C.), protected from light. The dye-conjugated antibody was purified by size exclusion chromatography. The degree of labeling (mole fluorophore/mole antibody), determined spectrally, was 1.6.

The dye conjugated antibody was adjusted to 1 mg/ml and filtered through an ELF® spin filter, 0.2 μm pore size, (E6606, Invitrogen Corp., Eugene, OR) that had been sanitized by prior filtration of 70% ethyl alcohol.

### Example 4

Visualization of a LS174T human colon adenocarcinoma tumor in a whole mouse using anti-CEA Ab-Alexa Fluor 750 Conjugate

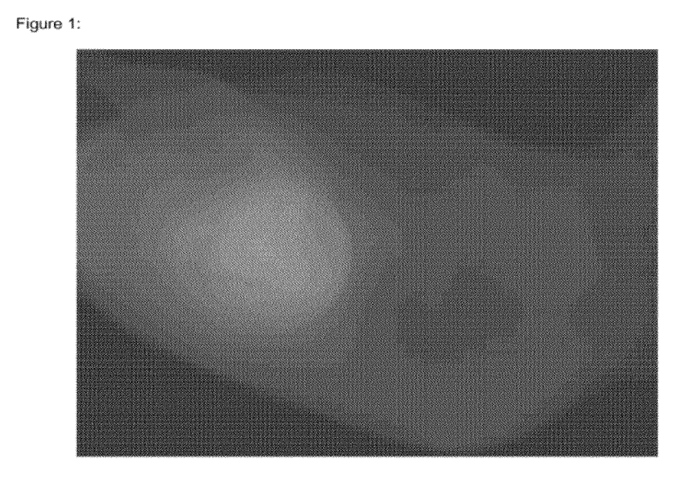

One female athymic nu/nu mouse was injected with one million LS174T human colorectal adenocarcinoma cells (ATCC CL-188) sub-cutaneous. When the tumor mass reached one centimeter in diameter and was visibly vascularized, t the tumors were visualized by injecting the mouse with anti-CEA-Alexa Fluor 750 dye antibody conjugate. The anti-CEA Ab-Alexa Fluor 750 dye conjugate was prepared according to Example 2 and used at a final protein concentration of 1.1 mg/ml and a DOL of 1.6. 50 μg of the anti-CEA Ab-Alexa Fluor 750 dye conjugate was injected into the mouse intravenously via the tail vein. The mouse was imaged with CRi Maestro Imaging System (Ex: 740 nm; Em: 790-950 nm) at 15 min, 30 min, 90 min, 2 hr, 6 hr, 24 hr, 48 hr, 3 days, 4 days and 7 days post-injection. The CEA Ab conjugate localized to the tumor mass and was visible in the whole mouse, See FIG. 1. No detectable accumulation was found in the liver or other organs.

### Example 5

Time course visualization of a LS174T human colon adenocarcinoma tumor in a whole mouse using anti-CEA Ab-Alexa Fluor 750 Dye Conjugate

As described in Example 4, one female athymic nu/nu mouse was injected with one million LS174T human colorectal adenocarcinoma cells (ATCC CL-188) sub-cutaneous. When the tumor mass reached one centimeter in diameter and was visibly vascularized, the tumors were visualized by injecting the mouse with anti-CEA-Alexa Fluor 750 dye antibody conjugate. The anti-CEA Ab-Alexa Fluor 750 dye conjugate was prepared according to Example 2 and used at a final protein concentration of 1.1 mg/ml and a DOL of 1.6. 50 μg of the anti-CEA Ab Alexa Fluor 750 dye conjugate was injected into the mouse intravenously via the tail vein. The mouse was imaged with CRi Maestro Imaging System (Ex: 735 nm; Em: 790-950 nm) at 1 hr, 2 hr, 6 hr, 24 hr, 48 hr, 3 days, 6 days, 7 days and 8 days post-injection. The CEA Ab conjugate localized to the tumor mass and was visible in the whole mouse. No detectable accumulation was found in the liver or other organs.

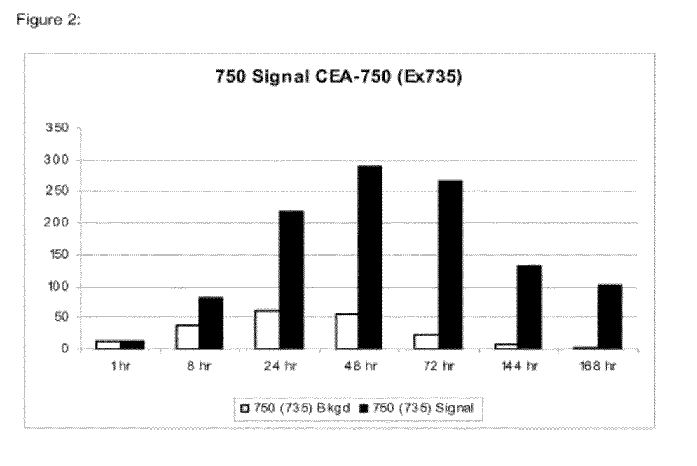

FIG. 2 shows a time course of tumor labeling by CEA Ab-Alexa Fluor 750 conjugate. Signal refers to average intensity of the labeling in the tumor; background refers to signal from the mouse body next to the tumor.

### Example 6

Visualization of a LS174T human colon adenocarcinoma tumor in a whole mouse using anti-CEA Ab-Alexa Fluor 790 Dye Conjugate

One female athymic nu/nu mouse was injected with one million LS174T human colorectal adenocarcinoma cells (ATCC CL-188) sub-cu. A second female athymic nu/nu mouse was injected with one million SW620 human colorectal adenocarcinoma cells (ATCC CCL-227) sub-cu. LS174T is a high CEA producer (1944 ng/1 million cells/10 days (ATCC)), while the SW620 is a low CEA producer (0.15 ng/1 million cells/10 days(ATCC)) and serves as a negative control. When the tumor masses reached five millimeters in diameter, the tumors were visualized by injecting the mouse with anti-CEA-Alexa Fluor 790 dye antibody conjugate. The CEA Ab-Alexa Fluor 790 conjugate was prepared according to Example 3 and used at a final protein concentration of 1.1 mg/ml and a DOL of 1.6. 50pg of the anti-CEA Ab-Alexa Fluor 790 dye conjugate was injected into each mouse IV intravenously via the tail vein. The mice were imaged with CRi Maestro Imaging System (Ex: 740 nm; Em: 790-950 nm) at 30 min, 1 hr, 2 hr, 24 hr, 48 hr, 6 days, 7 days, 8 days, 9 days and 10 days post-injection.

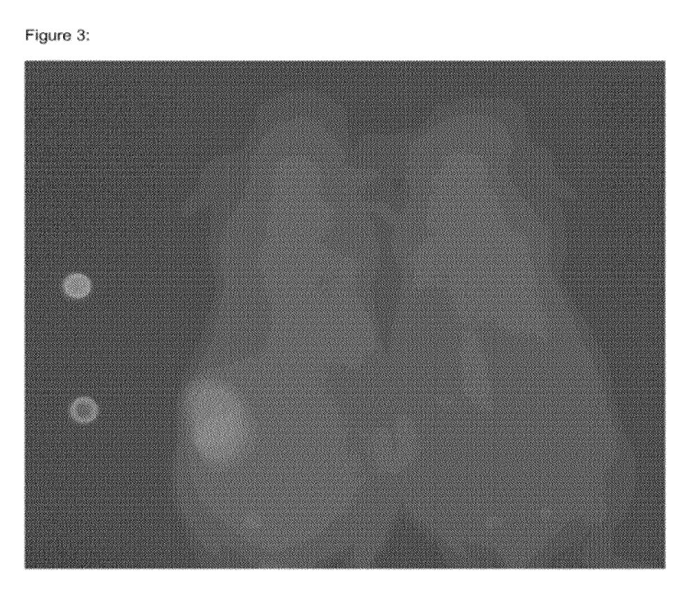

The anti-CEA Ab dye conjugate localized to the LS174T tumor mass but did not label the SW620 tumor, see FIG. 3, wherein the anti-CEA Ab dye conjugate is targeting to the CEA in the tumor; the labeling is not due to non-specific uptake. No detectable accumulation was found in the liver or other organs.

### Example 7

Multiplex: Vasculature+Tumor Marker

An athymic nu/nu mouse carrying an LS174T human colon adenocarcinoma xenograft was injected intravenously with 50 μg of anti-CEA-Alexa Fluor 750 dye antibody conjugate as described in Example 5. The antibody was prepared using the SAIVI Alexa Fluor® 750 Antibody/Protein Labeling Kit (Cat. no. S30040). Nine days after injection of the anti-CEA antibody conjugate, the animal was injected intravenously with SAIVI™ Alexa Fluor® 680 injectable contrast agent *human serum transferrin* (Cat. no. S34790) to highlight the tumor vasculature.

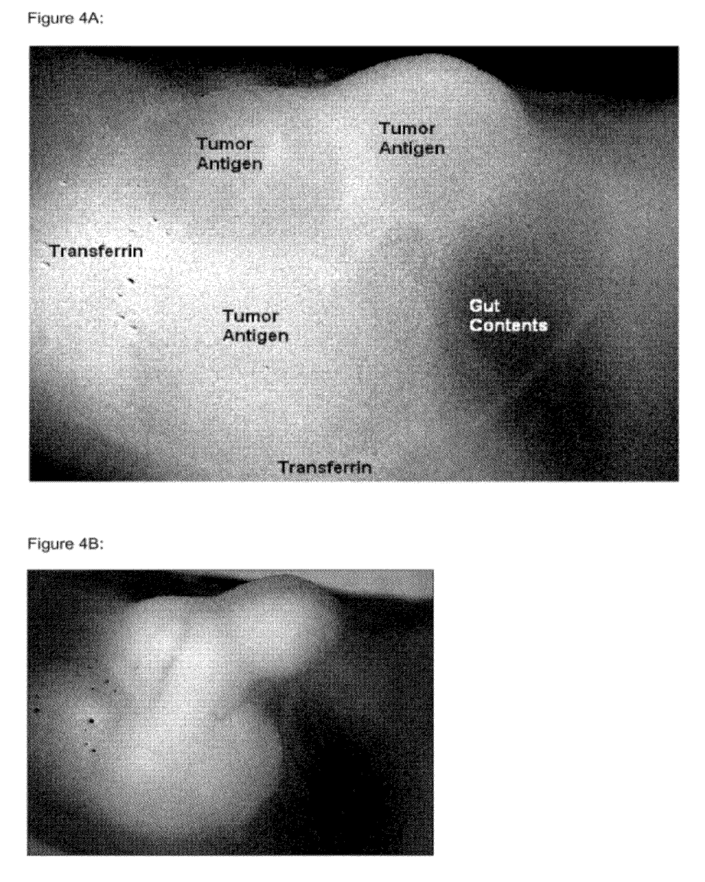

The animal was imaged 60 minutes after intravenous injection of the transferrin using the CRi Maestro system (687 nm excitation and 740-950 nm bandpass emission). Three distinct sources of fluorescence can be detected following spectral unmixing of the image cube using the Nuance™ Software. The transferrin (green), CEA antigen (red), and gut contents (blue) signals can be clearly separated. See FIG. 4.

The reagents employed in the examples are commercially available or can be prepared using commercially available instrumentation, methods, or reagents known in the art. The foregoing examples illustrate various aspects of the invention and practice of the methods of the invention. The examples are not intended to provide an exhaustive description of the many different embodiments of the invention. Thus, although the forgoing invention has been described in some detail by way of illustration and example for purposes of clarity of understanding, those of ordinary skill in the art will realize readily that many changes and modifications can be made thereto without departing from the spirit or scope of the appended claims.

All of the above-cited references are hereby incorporated by reference as if set forth fully herein.

1. A method for imaging a target antigen in a living body, wherein the method comprises;
a) providing a dye conjugate comprising a NIR dye and an antibody that binds to the target antigen;
b) introducing the dye conjugate into the body to form a contacted body;
c) illuminating the contacted body with an appropriate wavelength to form an illuminated body; and
d) observing the illuminated body wherein the target antigen is imaged;
wherein the target antigen is associated with cancer.

2. The method of claim 1, wherein the target antigen is CEA (Carcinoembryonic Antigen).

3. The method of claim 1, wherein the target antigen is selected from the group consisting of α₂-Macroglobulin, α-Fetoprotein (AFP), β₂-Microglobulin, β-Catenin, ACTH C terminal, ACTH N terminal, ACTR/AIB1, Alpha Fetoprotein, BCA-225, Bcl-2, BRCA2, Bromodeoxyuridine, CA 125, CA 15-3, CA 19-9, Calcitonin, Calretinin, Cathepsin D, CD15, CD63, CD74, CEA (Carcinoembryonic Antigen), Chorionic gonadotropin (β-subunit) (βHCG), Chromogranin A, c-Kit (CD 117), Cks1, Clathrin Antigen, Claudin-3, Claudin-4, Claudin-7, c-Met, c-Myc, Collagen Type IV, Collagen Type VII, COX-1, COX-2, Cyclin D1/D2 & D3, Cyclin E, Cytokeratin (Acidic or Basic), Cytokeratin (HMW), Cytokeratin 18, Cytokeratin 19, Cytokeratin 20, Cytokeratin ⅚, Cytokeratin 6, Cytokeratin 7, Cytokeratin 8, Cytokeratin 8/18, E2F-1, E-Cadherin, EGFr, EGP2 (Epithelial Glycoprotein 2), EMA (Epithelial Membrane Antigen), EMMPRIN, Enolase, EphB4 Receptor, ER (Estrogen Receptor), EZH2, Ezrin, FHIT, Galectin-1, Galectin-3, GCDFP-15, Glial Filament Acidic Protein, HER2 (c-erbB-2), HER4, HPV Early Protein, HPV16 Late I Protein, Human Epithelial Proliferating Ag, Human Epithelium Specific Ag, Human Milk Fat Globule Membrane, Human Milk Fat Globulin (HMFG1), Human Milk Fat Globulin (HMFG2), lnvolucrin, JAB1, Ki-67, Lewis A Ag, LRP/MVP, Major Vault Protein, MAP Kinase (ERK1+ERK2), MART-1 (Melan-A), MDM2, Melanoma Associated Antigen, Melanosome, Metallothionein, MGMT, MLH1, MSH2, MSH6, MTA1, MUC1 (Mucin 1), MUC2 (Mucin 2), MUC5AC, N-Cadherin, Neu-Oncogene, Nitric Oxide Synthase, Nucleophosmin/B23, NY-ESO-1, Occludin, p16, p21 (WAF1/Cip1), p27, p34, P53 Oncoprotein, Pancreatic Islet Cell Antibody, PARP, Paxillin, PD-ECGF, P-Giycoprotein (MDR), phospho-MAP Kinase (ERK1+2), Phosphotyrosine, Placental Alkaline Phosphatase, PR (Progesterone Receptor), PRL-3, PRLr, Proliferating Cell Protein Ki-67, pS2, PSA (Prostate Specific Antigen), PsAP (Prostatic Acid Phosphatase), PTEN, PTTG-1 (Pituitary Tumor Transforming Gene-1), Retinoblastoma Gene Product, SCLC (Small Cell Lung Cancer, CD56, N-CAM), Sialyl Lewis A, SKP2, Smad3, STAT3, TAG-72 (CA 72.4), TdT, Tenascin, Thyroglobulin, TIMP-2, Topo II, TS (Thymidylate Synthase), TTF-1 (Thyroid Transcription Factor 1), uPAR, Villine, Vmentin, Wtl (Wilm's tumor), and Z0-1.

4. The method of claim 1, wherein the introducing step is non-invasive such that the integrity of the body is not disrupted.

5. The method of claim 1, wherein the NIR dye has an excitation wavelength of about 580 nm to about 800 nm.

6. The method of claim 1, wherein the NIR dye has an excitation wavelength of about 660 nm to about 790 nm.

7. The method of claim 1, wherein the NIR dye is selected from the group consisting of a pyrene, an anthracene, a naphthalene, an acridine, a stilbene, an indole or benzindole, an xazole or benzoxazole, a thiazole or benzothiazole, a 4-amino-7-nitrobenz-2-oxa-1, 3-diazole (NBD), a carbocyanine, a carbostyryl, a porphyrin, a salicylate, an anthranilate, an azulene, a perylene, a pyridine, a quinoline, a borapolyazaindacene, a xanthene, an oxazine, a benzoxazine, a resorufin, a carbazine, a phenalenone, a coumarin, a benzofuran, a benzphenalenone and derivatives thereof.

8. The method of claim 1, wherein the NIR dye is a semiconductor nanocrystal.

9. The method of claim 1, wherein the NIR dye is impregnated in or associated with a microsphere.

10. The method of claim 1, wherein the antibody is a monoclonal antibody.

11. The method of claim 1, wherein the living body is a non-human vertebrate.

12. The method of claim 11, wherein the living body is a mouse or rat.

13. The method of claim 1, wherein the living body is a human.

14. The method of claim 1, wherein the cancer is cervical cancer, testicular tumor, lung carcinoma, small cell lung carcinoma, bladder carcinoma, epithelial carcinoma, glioma, colorectal carcinoma, pancreatic cancer, colon cancer, breast cancer, ovarian cancer, prostate cancer, squamous cell carcinoma, basal cell carcinoma, adenocarcinoma, renal cell carcinoma, hepatoma, bile duct carcinoma, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, rhabdosarcoma, sweat gland carcinoma, sebaceous gland carcinoma, papillary carcinoma, papillary adenocarcinomas, cystadenocarcinoma, medullary carcinoma, bronchogenic carcinoma, choriocarcinoma, seminoma, embryonal carcinoma, Wilms' tumor, astrocytoma, Kaposi's sarcoma, medulloblastoma, craniopharyngioma, ependymoma, pinealoma, hemangioblastoma, acoustic neuroma, oligodendroglioma, meningioma, neuroblastoma, retinoblastoma, myeloma, lymphoma, or leukemia.

15. The method of claim 1, wherein the introducing step comprises parenteral administration of the dye conjugate into the body.16.

16. The method of claim 1, further comprising a step of:
incubating the contacted body for a period of time sufficient for the dye conjugate to contact the target antigen.

17. The method of claim 1, wherein the period of time is at least 90 minutes.

18. The method of claim 1, further comprising a step of:
transmitting data onto a computer processor, wherein the data represents the illuminated body; and
performing an analysis of the data with the computer processor to determine a result indicating the presence, amount or location of the target antigen.

19. The method of claim 18, further comprising a display unit or printout which visually displays the result.

20-26. (canceled)
